(19) United States
(12) Patent Application Publication
(10) Pub. No.: US 2024/0033428 A1
(43) Pub. Date: Feb. 1, 2024

(54) PUMP-CONNECTABLE ANALYTE SENSING CANNULA

(71) Applicant: Pacific Diabetes Technologies Inc, Portland, OR (US)

(72) Inventors: Robert S. CARGILL, Portland, OR (US); Solomon REID, Portland, OR (US); William Kenneth WARD, Portland, OR (US); Thomas L. SEIDL, Wilsonville, OR (US)

(21) Appl. No.: 18/227,071

(22) Filed: Jul. 27, 2023

Related U.S. Application Data
(63) Continuation of application No. PCT/US22/14116, filed on Jan. 27, 2022.
(60) Provisional application No. 63/142,696, filed on Jan. 28, 2021.

(51) Int. Cl. A61M 5/172; A61B 5/1486; A61B 5/145; A61M 5/142

(52) U.S. Cl. CPC A61M 5/1723; A61B 5/14865; A61B 5/14532; A61M 5/14248; A61M 2005/14252; A61M 2005/1726

(57) ABSTRACT

Described herein are systems and methods for sensing an analyte concentration and delivering a composition to a subcutaneous space of a subject comprising a sensing cannula, which comprises an electrode for detecting the analyte concentration in the subcutaneous space.

### CROSS-REFERENCE

This application is a continuation of International Application No. PCT/US2022/014116, filed Jan. 27, 2022, which claims the benefit of U.S. Provisional Application No. 63/142,696, filed Jan. 28, 2021, each of which is incorporated by reference herein in its entirety.

### BACKGROUND

There are several medical therapies that involve treatment through the subcutaneous infusion of fluid. Glatiramer acetate, a treatment for multiple sclerosis, may be prescribed for daily subcutaneous injection. Heparin may also be administered via frequent subcutaneous injection as a treatment for certain clotting disorders. Human menopausal gonadotropin is injected subcutaneously on a daily basis in women underdoing fertility treatments. Further, pediatric patients undergoing parenteral nutrition supplementation may also receive repeated subcutaneous doses of multivitamins. Subcutaneous injections are also becoming more common in veterinary applications.

One of the largest populations on daily subcutaneous injections is the group of people with insulin-treated Type 1 or Type 2 diabetes mellitus. Many of these individuals use a pump to deliver insulin on a continuous basis into the subcutaneous space, a process known as continuous subcutaneous insulin infusion (CSII). Many patients who use CSII deliver the insulin by using a pump worn directly on the skin. These pumps are known as patch pumps.

In many instances, it is desirable to both track the concentration of an analyte and deliver a medication in response to the level of the analyte. This is particularly true of glucose and insulin, as state of the art insulin pumps now include automated insulin dosing based upon readings from a CGM sensor. For the convenience of the user, it would be desirable to combine both sensing and infusion into a single device. However, despite the availability of both CGM sensors and infusion ports for nearly two decades, there has been little progress toward combining the two functions into a single unified device. Consequently, automated insulin dosing pumps continue to use physically separated sensors and infusion sites. This multiplicity of sites requires additional time to manage, increases pain and infection risk, and increases cost to the patient.

In the case of glucose measurement, one factor that has prevented integration is the assumption by those in the field of diabetes that insulin delivery in proximity to a glucose sensor would corrupt sensor readings due to local interference with the analyte. The orthodoxy holds that there should be separation between the site of insulin delivery and glucose monitoring. For example, in Dexcom's G6 instructions it instructs the user to “choose a site at least 3 inches from insulin pump infusion set or injection site” (p. 11, Dexcom G6 User Guide, 2017), incorporated herein by reference in its entirety. Further, Abbott says to keep its Libre sensor “at least 1 inch away from an insulin injection site” (p. 21 Libre In-Service Guide, Abbott ADC-05821 v2.0 10/17) and Medtronic advises “1 inch from your insulin pump infusion site” and “1 inch from any manual insulin injection site.” (p. 12, My Guardian Connect manual, Medtronic 27 Apr. 2018), each incorporated herein by reference in its entirety. Moreover, every insertion site involves piercing the skin with a separate needle that may be painful for the patient, and each site brings with it the risk of complications such as scarring and infection. The physical separation and resulting complexity can also increase the cost and size of the device worn on the body.

### SUMMARY

Provided herein is a unified analyte sensing fluid delivery cannula for both analyte sensing and fluid delivery to a subject. This can provide a less painful, less expensive, more discreet, and more convenient experience for a subject. In some embodiments, the fluid is delivered to the subcutaneous tissue of the subject. In some embodiments, an analyte sensor is directly disposed on the surface of the cannula since the physiological effect of an analyte on surrounding subcutaneous tissue analyte concentration can be negligible. In some instances, electroactive components of a composition excipient (e.g., insulin excipient) can interfere with an analyte sensor (e.g., amperometric glucose sensors) that cause the sensor current to initially rise, followed by a permanent loss of analyte sensitivity. However, interstitial fluid analyte levels can be measured in the immediate vicinity of a composition delivery (e.g., insulin delivery) site through the use of an appropriately designed sensor (e.g., amperometric glucose sensors), for example, as described in U.S. Pat. No. 10,780,222, which is incorporated herein by reference in its entirety. In some embodiments, an infusion device comprising the unified analyte sensing and fluid delivery cannula can enable the simultaneous connection of an amperometric sensor on the surface of the cannula to signal processing electronics. In some cases, the infusion devices can further enable the simultaneous connection of an amperometric sensor on the surface of the cannula to various pre-existing fluid (e.g., drug, composition, etc.) delivery mechanisms. These delivery mechanisms may comprise syringes, pens, or pumps, which are connected to the fluid path of the same infusion cannula.

In one aspect, disclosed herein are systems comprising a device for sensing an analyte concentration and delivering a composition to a subcutaneous space, comprising: a sensing cannula comprising an electrode for detecting the analyte concentration in the subcutaneous space, wherein the electrode comprises a redox-catalytic layer; a signal processing module operably coupled to the sensing cannula through a plurality of conductors; and a fluid reservoir configured to deliver the composition through the sensing cannula to the subcutaneous space upon receiving a signal from the signal processing module. In some embodiments, the device further comprises a housing having a top surface, a base, and one or more vertical walls. In some embodiments, the housing further comprises one or more electrical contacts on the one or more vertical walls. In some embodiments, one or more of the plurality of conductors is contained in the housing. In some embodiments, one or more of the plurality of conductors is on an external surface of the one or more vertical walls of the housing. In some embodiments, the sensing cannula comprises one or more electrical contacts. In some embodiments, the one or more of the plurality of conductors is in contact with the one or more electrical contacts on the sensing cannula. In some embodiments, one or more of the plurality of conductors is on an exterior surface of the sensing cannula. In some embodiments, one or more of the plurality of conductors is operably coupled to the electrode. In some embodiments, one or more of the plurality of conductors is held in contact with the sensing cannula through a connector. In some embodiments, the connector is an adhesive, an adhesive conductor, or a plastic retaining clip. In some embodiments, the device further comprises a fluid path connecting the fluid reservoir and the sensing cannula. In some embodiments, the fluid path is about 5 mm to about 10 mm in length. In some embodiments, the redox-catalytic layer comprises a redox mediator and an enzyme. In some embodiments, the redox mediator comprises a metal in the platinum group that is covalently bound to a pyridine-based or imidazole-based ligand. In some embodiments, the enzyme comprises glucose oxidase or glucose dehydrogenase. In some embodiments, the redox mediator and the enzyme allow electron transfer from an analyte in the subcutaneous space to the electrode. In some embodiments, the electron transfer is sufficient to cause a response to an applied bias potential of no more than +250 millivolts (mV) relative to a reference electrode. In some embodiments, the response comprises a current. In some embodiments, the applied bias potential of no more than +250 mV relative to the reference electrode allows the electrode layer to undergo substantially no electropolymerization of the excipient during continuous operation of the electrode, thereby maintaining a sensitivity to analyte in the presence of the composition. In some embodiments, the electrode is an amperometric glucose sensor. In some embodiments, the analyte is glucose. In some embodiments, the composition comprises insulin or an insulin analog. In some embodiments, the composition comprises fast-acting insulin, intermediate-acting insulin, or long-acting insulin. In some embodiments, the composition further comprises at least one pharmaceutical acceptable excipient. In some embodiments, the at least one pharmaceutical acceptable excipient comprises phenol, cresol, a salts, a stabilizing agent, or any combination thereof. In some embodiments, the plurality of conductors comprise a conductive metal or alloy. In some embodiments, the conductive metal or alloy is copper, brass or steel. In some embodiments, the plurality of conductors comprise a compressible conductive rubber. In some embodiments, the plurality of conductors comprise a flexible electronic circuit. In some embodiments, the flexible electronic circuit comprises a polyimide film supporting traces formed from patterned copper foil. In some embodiments, the device further comprises a spring to drive the sensing cannula into the subcutaneous space. In some embodiments, the spring applies force against a sliding clip comprising the sensing cannula.

In another aspect, disclosed herein are systems comprising devices for sensing analyte concentration and delivering a composition to a subcutaneous space, the device comprising: a sensing cannula comprising an electrode for detecting an analyte concentration in the subcutaneous space; a signal processing module operably coupled to the sensing cannula through a plurality of conductors; and an electronic pump configured to depress a syringe configured to a fluid reservoir upon receiving a signal from the signal processing module, wherein depressing the syringe delivers the composition through the sensing cannula to the subcutaneous space. In some embodiments, the device further comprises a housing having a top surface, a base, and one or more vertical walls. In some embodiments, the housing further comprises one or more electrical contacts on the one or more vertical walls. In some embodiments, one or more of the plurality of conductors is contained in the housing. In some embodiments, one or more of the plurality of conductors is on an external surface of the one or more vertical walls of the housing. In some embodiments, the sensing cannula comprises one or more electrical contacts. In some embodiments, the one or more of the plurality of conductors is in contact with the one or more electrical contacts on the sensing cannula. In some embodiments, one or more of the plurality of conductors is on an exterior surface of the sensing cannula. In some embodiments, one or more of the plurality of conductors is operably coupled to the electrode. In some embodiments, one or more of the plurality of conductors is held in contact with the sensing cannula through a connector. In some embodiments, the connector is an adhesive, an adhesive conductor, or a plastic retaining clip. In some embodiments, the device further comprises a fluid path connecting the fluid reservoir and the sensing cannula. In some embodiments, the fluid path is about 5 mm to about 10 mm in length. In some embodiments, the electrode comprises a redox-catalytic layer. In some embodiments, the redox-catalytic layer comprises a redox mediator and an enzyme. In some embodiments, the redox mediator comprises a metal in the platinum group that is covalently bound to a pyridine-based or imidazole-based ligand. In some embodiments, the enzyme comprises glucose oxidase or glucose dehydrogenase. In some embodiments, the redox mediator and the enzyme allow electron transfer from an analyte in the subcutaneous space to the electrode. In some embodiments, the electron transfer is sufficient to cause a response to an applied bias potential of no more than +250 millivolts (mV) relative to a reference electrode. In some embodiments, the response comprises a current. In some embodiments, the applied bias potential of no more than +250 mV relative to the reference electrode allows the electrode layer to undergo substantially no electropolymerization of the excipient during continuous operation of the electrode, thereby maintaining a sensitivity to analyte in the presence of the composition. In some embodiments, the electrode is an amperometric glucose sensor. In some embodiments, the analyte is glucose. In some embodiments, the composition comprises insulin or an insulin analog. In some embodiments, the composition comprises fast-acting insulin, intermediate-acting insulin, or long-acting insulin. In some embodiments, the composition further comprises at least one pharmaceutical acceptable excipient. In some embodiments, the at least one pharmaceutical acceptable excipient comprises phenol, cresol, a salts, a stabilizing agent, or any combination thereof. In some embodiments, the plurality of conductors comprise a conductive metal or alloy. In some embodiments, the conductive metal or alloy is copper, brass or steel. In some embodiments, the plurality of conductors comprise a compressible conductive rubber. In some embodiments, the plurality of conductors comprise a flexible electronic circuit. In some embodiments, the flexible electronic circuit comprises a polyimide film supporting traces formed from patterned copper foil. In some embodiments, the device further comprises a spring to drive the sensing cannula into the subcutaneous space. In some embodiments, the spring applies force against a sliding clip comprising the sensing cannula.

In another aspect, disclosed herein are methods for sensing an analyte concentration and delivering a composition to a subcutaneous space in a subject to treat a disease or disorder, comprising: providing a sensing cannula, wherein the sensing cannula comprises an electrode for detecting the analyte concentration in the subcutaneous space of the subject, wherein the electrode comprises a redox-catalytic layer; inserting the sensing cannula into the subcutaneous space; monitoring a behavior of the analyte concentration in the subcutaneous space; and delivering a sufficient amount of the composition through the sensing cannula into the subcutaneous space in response to the behavior of the analyte concentration. In some embodiments, the disease or disorder comprises insulin resistance. In some embodiments, the disease or disorder comprises Type 1 diabetes mellitus. In some embodiments, the disease or disorder comprises Type 2 diabetes mellitus. In some embodiments, detecting the analyte concentration comprises measuring an applied bias potential of the electrode relative to a reference electrode. In some embodiments, the redox-catalytic layer comprises a redox mediator and an enzyme. In some embodiments, the redox mediator comprises a metal in the platinum group that is covalently bound to a pyridine-based or imidazole-based ligand. In some embodiments, the enzyme comprises glucose oxidase or glucose dehydrogenase. In some embodiments, the redox mediator and the enzyme allow electron transfer from an analyte in the subcutaneous space to the electrode. In some embodiments, the electron transfer is sufficient to cause a response to an applied bias potential of no more than +250 millivolts (mV) relative to a reference electrode. In some embodiments, the response comprises a current. In some embodiments, the applied bias potential of no more than +250 mV relative to the reference electrode allows the electrode layer to undergo substantially no electropolymerization of the excipient during continuous operation of the electrode, thereby maintaining a sensitivity to analyte in the presence of the composition. In some embodiments, the electrode is an amperometric glucose sensor. In some embodiments, the analyte is glucose. In some embodiments, the composition comprises insulin or an insulin analog. In some embodiments, the composition comprises fast-acting insulin, intermediate-acting insulin, or long-acting insulin. In some embodiments, the composition further comprises at least one pharmaceutical acceptable excipient. In some embodiments, the at least one pharmaceutical acceptable excipient comprises phenol, cresol, a salts, a stabilizing agent, or any combination thereof. In some embodiments, the sensing cannula is operably coupled to a signal processing module through a plurality of conductors. In some embodiments, the signal processing module sends a signal to a fluid reservoir comprising the composition. In some embodiments, the fluid reservoir is configured to deliver the composition through the sensing cannula to the subcutaneous space upon receiving a signal from the signal processing module. In some embodiments, the behavior of the analyte concentration comprises an analyte concentration reaching a threshold. In some embodiments, the behavior of the analyte concentration comprises a duration that the analyte concentration remains at a threshold. In some embodiments, the behavior of the analyte concentration comprises a rate of change in the analyte concentration. In some embodiments, the rate of change in the analyte concentration comprises an increase in the analyte concentration. In some embodiments, the rate of change in the analyte concentration comprises a decrease in the analyte concentration.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1A shows a non-limiting example of a perspective view of an integrated sensing cannula assembly with the top surface of the assembly sectioned, in accordance with some embodiments;

FIG. 1B shows a non-limiting example of a perspective view of an integrated sensing cannula assembly with the top surface of the assembly removed, in accordance with some embodiments;

FIG. 2A shows a non-limiting example of a perspective view of an integrated sensing cannula assembly aligned to a mating patch pump prior to insertion, in accordance with some embodiments;

FIG. 2B shows a non-limiting example of a sectional view of an integrated sensing cannula assembly in its final position, in accordance with some embodiments;

FIG. 2C shows a non-limiting example of a close-up view of an integrated sensing cannula assembly in its final position, in accordance with some embodiments;

FIG. 2D shows a non-limiting example of a side section of an integrated sensing cannula assembly in its final position, in accordance with some embodiments;

FIG. 3A shows a non-limiting example of a side sections of an integrated sensing cannula assembly retained within a spring-loaded insertion device aligned to a mating patch pump prior to insertion, in accordance with some embodiments;

FIG. 3B shows a non-limiting example of a side sections of an integrated sensing cannula assembly retained within a spring-loaded insertion device aligned to a mating patch following insertion, in accordance with some embodiments;

FIG. 4A shows a non-limiting example of a perspective view of an integrated sensing cannula assembly aligned to a mating patch pump with permanent fluid path and electrical connections, prior to deployment, in accordance with some embodiments;

FIG. 4B shows a non-limiting example of a perspective view of an integrated sensing cannula assembly aligned to a mating patch pump with permanent fluid path and electrical connections, following deployment, in accordance with some embodiments;

FIG. 5A shows a non-limiting example of a perspective view of an integrated sensing cannula assembly having an integrated inserter mechanism fitted within a patch pump, prior to insertion, in accordance with some embodiments;

FIG. 5B shows a non-limiting example of a top views of an integrated sensing cannula assembly having an integrated inserter mechanism fitted within a patch pump, prior to insertion, in accordance with some embodiments;

FIG. 5C shows a non-limiting example of close-up, sectional view of an integrated sensing cannula assembly having an integrated inserter mechanism placed within a patch pump, following insertion, in accordance with some embodiments;

FIG. 5D shows a non-limiting example of perspective view of an integrated sensing cannula assembly having an integrated inserter mechanism, fitted within a patch pump, following insertion, in accordance with some embodiments;

FIG. 6A shows a non-limiting example of a side view of a sensing cannula having electrical and fluid connections, in accordance with some embodiments; and

FIG. 6B shows a non-limiting example of a perspective view of a sensing cannula having electrical and fluid connections, in accordance with some embodiments.

### DETAILED DESCRIPTION

Described herein, in certain embodiments, is a combined subcutaneous fluid delivery (e.g., composition, drug, medication, etc.) and amperometric analyte sensing system that is suitable for integration with various body-worn pumps. In some embodiments, the system comprises an interoperable assembly that includes a combined sensor and cannula (e.g., sensing cannula) attached to or within a housing. In some cases, the system further comprises electrical coupling of the sensing cannula to a signal processing module. In some cases, the system further comprises fluidic coupling of the sensing cannula to a fluid reservoir comprising a composition, drug, medication, or any suitable fluid for delivery to a subject.

Described herein, in certain embodiments, is a device for sensing an analyte concentration and delivering a composition to a subcutaneous space. In some embodiments, the device comprises a sensing cannula comprising an electrode for detecting the analyte concentration in the subcutaneous space. In some instances, the electrode is on an external surface of the sensing cannula. In some cases, the electrode comprises a redox-catalytic layer. In some embodiments, the device further comprises a signal processing module operably coupled to the sensing cannula through a plurality of conductors. In some embodiments, the device comprises a fluid reservoir configured to deliver the composition through the sensing cannula to the subcutaneous space upon receiving a signal from the signal processing module.

Also described herein, in certain embodiments, is a device for sensing an analyte concentration and delivering a composition to a subcutaneous space. In some embodiments, the device comprises a sensing cannula comprising an electrode for detecting an analyte concentration in the subcutaneous space. In some instances, the electrode is on an external surface of the sensing cannula. In some embodiments, the device further comprises a signal processing module operably coupled to the sensing cannula through a plurality of conductors. In some embodiments, the device comprises an electronic pump configured to depress a syringe configured to a fluid reservoir upon receiving a signal from the signal processing module. In some cases, depressing the syringe delivers the composition through the sensing cannula to the subcutaneous space.

Further described herein, in certain embodiments, is a method for sensing an analyte concentration and delivering a composition to a subcutaneous space in a subject to treat a disease or disorder. In some embodiments, the method comprises providing a sensing cannula. In some cases, the sensing cannula comprises an electrode for detecting the analyte concentration in the subcutaneous space of the subject. In some instances, the electrode is on an external surface of the sensing cannula. In some instances, the electrode comprises a redox-catalytic layer. In some embodiments, the method comprises inserting the sensing cannula into the subcutaneous space of a subject. In some cases, the method comprises monitoring a behavior of the analyte concentration in the subcutaneous space. In some embodiments, the method further comprises delivering a sufficient amount of the composition through the sensing cannula into the subcutaneous space in response to the behavior of the analyte concentration.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The terms “coupled” and “connected,” along with their derivatives, may be used herein. It should be understood that these terms are not intended as synonyms for each other. Rather, in particular embodiments, “connected” may be used to indicate that two or more elements are in direct physical or electrical contact with each other. “Coupled” may be used to indicate that two or more elements are in direct physical or electrical contact. However, “coupled” may also be used to indicate that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

As used herein, the term “tube”, “cannula”, or “passageway” may refer to a hollow cylinder that can be used for transporting or delivery a composition (e.g., insulin composition, such as those described herein). In some examples, the tube comprises a solid wall, a porous wall (e.g., with passages), or a combination thereof.

With respect to the use of any plural and/or singular terms herein, the plural can be translated to the singular and/or the singular can be translated to the plural, as is appropriate to the context and/or application. The various singular/plural permutations may be expressly set forth herein for sake of clarity.

Various operations may be described as multiple discrete operations in turn, in a manner that may be helpful in understanding embodiments; however, the order of description should not be construed to imply that these operations are order-dependent.

The description may use perspective-based descriptions such as “up”, “down”, “back”, “front”, “top” and “bottom”. Such descriptions are merely used to facilitate the discussion and are not intended to restrict the application of disclosed embodiments.

The present disclosure provides systems and methods for a combined subcutaneous fluid delivery and analyte sensing system in a form suitable for integration with various body-worn pumps. In some embodiments, the system is an infusion device adapted for use having a sensor and a cannula on a single component. In some embodiments, the system can be used for in vivo monitoring of an analyte concentration including, but not limited to, pH, oxygen, lactate, glucose, or insulin concentration, and delivery of a composition, drug, or medication including but not limited to glatiramer acetate, heparin, human menopausal gonadotropin, insulin, an insulin analog, a vitamin, a nutrient supplement, or any combination thereof. In some cases, the composition comprises fast-acting insulin, intermediate-acting insulin, long-acting insulin, or any combination thereof. In some embodiments, the composition further comprises at least one pharmaceutical acceptable excipient. In some cases, the at least one pharmaceutical acceptable excipient comprises phenol, cresol, a salts, a stabilizing agent, or any combination thereof. In some embodiments, analyte concentration monitoring can have applications in various situations including, but not limited to, treatment of multiple sclerosis, fertility treatments, diabetes, nutritional supplementation, and automated drug dosing.

The system of the present disclosure can be configured to be attached to the skin surface of a subject. In some embodiments, the infusion device is attached to the skin surface of the abdomen of the subject. In some embodiment, the infusion device comprises a single combined sensing cannula penetrating the skin surface and into the subcutaneous space of the subject for analyte sensing and fluid delivery. In some embodiments, the infusion device comprises an infusion pump worn on the body (e.g., abdomen).

In some embodiments, the device of the present disclosure can be about 1 square inch to about 3 square inches. In some examples, the device can be about 1 square inch to about 2 square inches, about 1 square inch to about 3 square inches, or about 2 square inches to about 3 square inches. In some examples, the device can be about 1 square inch, about 2 square inches, or about 3 square inches. In some examples, the device can be at least about 1 square inch, or about 2 square inches. In some examples, the device can be at most about 2 square inches, or about 3 square inches. In some cases, the device has a width of about 1.5 inches, a length of about 2 inches, and a height of about 0.5 inches. In some cases, the device is about 1.5 cubic inches. In some embodiments, the smaller size can provide an advantage over commercially available devices, such as those previously described. For instance, instead of requiring two separate devices on the body or two separate devices in one assembly, which sets a practical lower bound on the size of the device, the present disclosure describes devices comprising a single component attached to the skin.

Another advantage afforded by the infusion devices of the present disclosure comprises combining the sensor and cannula on a single component (e.g., sensing cannula), thereby only requiring one insertion needle in the subcutaneous tissue of the subject. In some embodiments, by avoiding the use of multiple insertion needles, the overall cost and waste of insertion devices can be reduced. In some embodiments, this also enables the subject to save valuable time. In some embodiments, reducing the number of insertion needles implies that the unified sensing cannula should be inserted to the subcutaneous space without damaging the fluid and electrical connections. In some cases, this can create constraints on the fluid and electrical connections.

Further, the infusion devices of the present disclosure enables the handling of electrical and fluid path connections to a combined sensor and fluid delivery cannula. In some embodiments, the electrical and fluid path connections are designed in the context of an interoperable assembly. In some cases, this design can mitigate the challenges associated with a combined sensor and a fluid delivery device, such as 1) the additional size and complexity added by an electronic interface, 2) challenges associated with co-location of electrical and fluid-handling features on a single percutaneous device, or 3) leakages of fluid into the electrical interface that can interfere with the ability of the sensor to record signal currents accurately.

In some embodiments, the infusion device is designed for mating with a patch pump (e.g., body-worn patch pump). In some cases, the patch pump can be used on the skin of a mammal (e.g., human) to supply a fluid. In some examples, the fluid comprises a composition, a drug, or a medication. In some embodiments, the patch pump comprises a cannula for delivering the fluid into the skin of the mammal. In some embodiments, the patch pump comprises a pumping mechanism to drive the fluid through the cannula. In some embodiments, the patch pump comprises a signal processing module.

In some embodiments, one or more of the components (e.g., cannula, fluid reservoir, signal processing module, conductor, etc.) in the infusion device of the present disclosure can be contained within a housing. In some cases, the housing comprises a top surface, a base, and one or more vertical walls. In some cases, the housing comprises a cylindrical shape with one or more electrical contacts on the vertical walls. In some embodiments, the integrated sensing cannula assembly is contained within a narrow cylindrically shaped housing. In some embodiments, this housing comprises an integrated inserter mechanism for driving the sensing cannula into the skin.

Interoperable Sensing Cannula

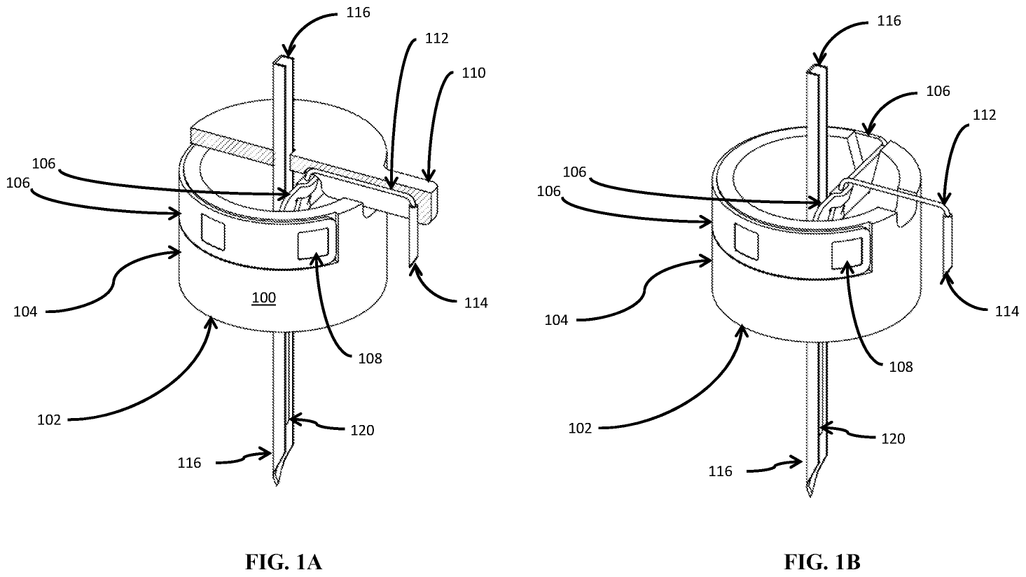

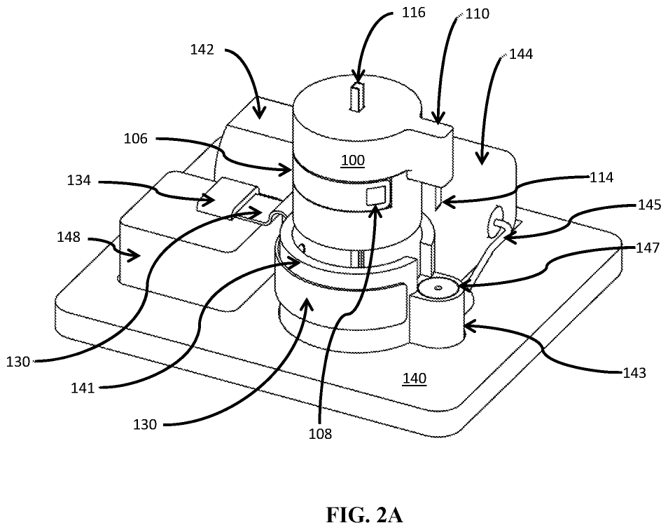

In some embodiments, the system comprises an interoperable assembly that includes a combined sensor and delivery cannula attached to a housing, which is exemplary illustrated in FIG. 1. In some cases, the cannula comprises a hollow tube fabricated of a polymer, a metal, or a combination thereof. In some instances, the cannula is fabricated from polyethylene, polyesters, epoxy, or any combination thereof. In some cases, the cannula comprises a stainless steel fluid path. In some instances, the cannula is about 0.5 mm to about 7 mm in length. In some instances, the cannula is about 0.5 mm to about 1 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 7 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 2 mm to about 3 mm, about 2 mm to about 4 mm, about 2 mm to about 5 mm, about 2 mm to about 6 mm, about 2 mm to about 7 mm, about 3 mm to about 4 mm, about 3 mm to about 5 mm, about 3 mm to about 6 mm, about 3 mm to about 7 mm, about 4 mm to about 5 mm, about 4 mm to about 6 mm, about 4 mm to about 7 mm, about 5 mm to about 6 mm, about 5 mm to about 7 mm, or about 6 mm to about 7 mm in length. In some instances, the cannula is about 0.5 mm, about 1 mm, about 2 mm, about 3 mm, about 4 mm, about 5 mm, about 6 mm, or about 7 mm in length. In some instances, the cannula is at least about 0.5 mm, about 1 mm, about 2 mm, about 3 mm, about 4 mm, about 5 mm, or about 6 mm in length. In some instances, the cannula is at most about 1 mm, about 2 mm, about 3 mm, about 4 mm, about 5 mm, about 6 mm, or about 7 mm in length. In some instances, the cannula comprises a microneedle. In some embodiments, the cannula comprises an analyte sensor on the exterior surface (e.g., an electrode or a plurality of electrodes) and one or more fluid delivery channels within, which can be referred to as a sensing cannula. In some embodiments, the sensing cannula is retained within a housing that provides a consistent electrical and fluid path interface for connection to a source of fluid (e.g., a fluid reservoir) and signal processing electronics (e.g., signal processing module).

Referring to FIG. 1A and FIG. 1B, a perspective view of an embodiment of the interoperable sensing cannula assembly is exemplary illustrated. In some embodiments, the assembly is configured for electrical and fluid path contacts to be established during placement of the assembly on the skin. The sensing cannula assembly 100 contains a sensing cannula 120 positioned within an inserter needle 116. In some embodiments, the sensing cannula assembly 100 can be contained in a cylindrical housing 102. In alternative embodiments, the sensing cannula assembly 100 can be contained in a non-cylindrical housing.

In some embodiments, the cylindrical housing 102 comprises one or more electrical conductors 106. In some cases, the one or more electrical conductors 106 are disposed in a circumferential orientation around the exterior surface of the housing on the side of the housing surface 104. These conductors 106 can be formed of conductive metals or alloys, including, but not limited to, copper, brass, or steel. In some examples, the conductors 106 may also be provided by a single or multi-conductor flexible electronic circuit, such as, but not limited to, a polyimide film supporting traces formed from patterned copper foil.

As illustrated in FIG. 1A and FIG. 1B, the top of the assembly can include a fluid path arm 110 that projects radially from the top and contains a fluid path tube 112. In some cases, the fluid path is connected to a fluid reservoir in order to deliver fluid to the sensing cannula 120. In some cases, the fluid path is composed of a flexible polymer. In some examples, the flexible polymer comprises a thermoplastic. In some examples, the flexible polymer comprises polytetrafluoroethylene (PTFE), fluorinated ethylene propylene (FEP), or any combination thereof. In some examples, a polymer, such as PTFE, FEP, or a combination thereof, can line the inside of a tubing composed of a thermoplastic polymer. In some cases, the fluid path tube 112 extends through the sensing cannula 120 and is placed in the subcutaneous tissue using a rigid inserter element (e.g., the inserter needle 116) or trocar that is removed immediately following insertion.

In some embodiments, the fluid path tube 112 terminates in a needle 114 configured essentially parallel to the axis of the sensing cannula assembly 100 in order to pierce a septum during insertion. In some embodiments, the fluid path tube 112 enters the sensing cannula 120 at the top or proximal end of the cannula, and is continuous with the hollow core of the cannula to allow fluid to flow from the fluid path tube 112 into the cannula 120. In some cases, the fluid path tube is about 2 mm to about 15 mm in length. In some cases, the fluid path is about 2 mm to about 5 mm, about 2 mm to about 7 mm, about 2 mm to about 10 mm, about 2 mm to about 12 mm, about 2 mm to about 15 mm, about 5 mm to about 7 mm, about 5 mm to about 10 mm, about 5 mm to about 12 mm, about 5 mm to about 15 mm, about 7 mm to about 10 mm, about 7 mm to about 12 mm, about 7 mm to about 15 mm, about 10 mm to about 12 mm, about 10 mm to about 15 mm, or about 12 mm to about 15 mm in length. In some cases, the fluid path is about 2 mm, about 5 mm, about 7 mm, about 10 mm, about 12 mm, or about 15 mm in length. In some cases, the fluid path is at least about 2 mm, about 5 mm, about 7 mm, about 10 mm, or about 12 mm in length. In some cases, the fluid path is at most about 5 mm, about 7 mm, about 10 mm, about 12 mm, or about 15 mm in length.

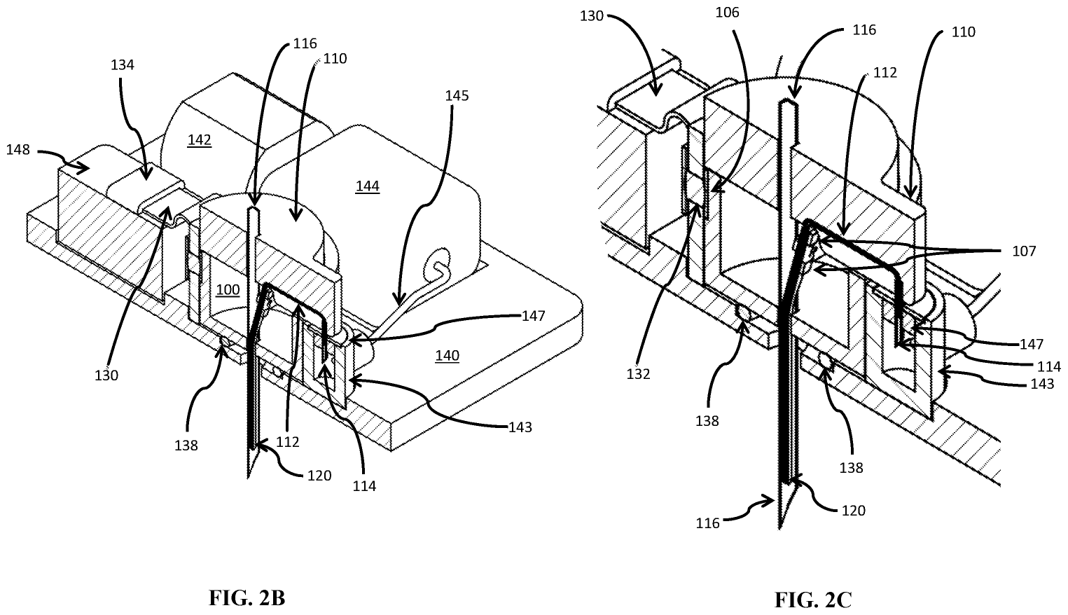

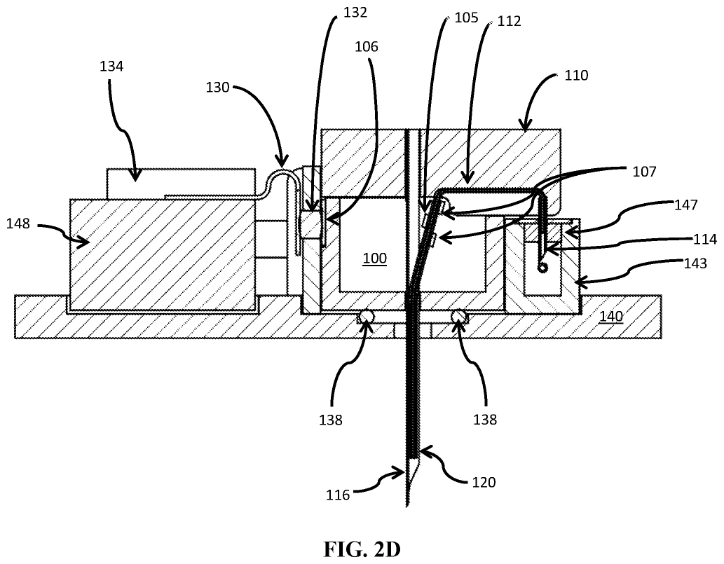

In some embodiments, the one or more conductors 106 also enters the center of assembly 100 and terminates in one or more contacts 107 within the assembly (shown in FIG. 2C and FIG. 2D) that are in electrical contact with a conductor on the cannula 120. The one or more conductors 106 can comprise one or more electrical contacts 108 on the exterior surface of the assembly to allow for electrical contact to an signal processing module configured to receive a signal (e.g., current) from the sensing cannula 120.

The interoperable sensing cannula assembly 100 can further be positioned within a pump base 140, as illustrated in FIG. 2A. The system of FIG. 2A illustrates a perspective view of the sensing cannula assembly 100 positioned within a pump base 140 prior to insertion into the skin. In some embodiments, the pump base 140 includes an alignment feature 141 that aligns the assembly 100 to the base 140 during insertion. In some examples, the alignment feature 141 is a ring. In alternative examples, the alignment feature 141 comprises one or more edges (e.g., as in a square, rectangle, triangle, etc.).

In some embodiments, the pump base 140 includes one or more conductors 130 with contact points configured to present a conductive surface to mating contacts 108 on the sensing cannula assembly 100. These one or more conductors 130 may be formed of conductive metals or alloys including, but not limited to, copper, brass, or steel. Alternatively, one or more conductors 130 may be provided by a single or multi-conductor flexible electronic circuit, such as, but not limited to, a polyimide film supporting traces formed from patterned copper foil. These conductors 130 can allow electrical contact to the sensing cannula 120 from an electronic signal processing module 148.

### Signal Processing Module

In some embodiments, an electronic signal processing module 148 is configured for electrical coupling to the sensor (e.g., sensing cannula 120). In some cases, the signal processing module 148 is configured for electronic processing of the sensor signals (e.g., current). In some instances, the sensor signals are signals from an electrode on the sensing cannula in a subcutaneous space. In some cases, the electronic signal processing module 148 facilitates the electromechanical interface between the sensor contacts and signal processing hardware. As illustrated in FIG. 2A, contact can be made to the electronic signal processing module 148 in component 134. In some examples, this component 134 can comprise a flex circuit socket. In some cases, the signal processing module enables the temporary or permanent electrical connection between sensor and associated processing electronics, and can permit the reuse of the electronics if desired.

In some embodiments, the signal from the sensor consists of at least one electrical current which is proportional to an analyte concentration in the subcutaneous space. In some examples, the electrical current is proportional to a glucose concentration in the subcutaneous space. In some embodiments, the electrical connection between the sensor and signal processing module 148 allows the signal processing module 148 to convert current into a value representing the analyte concentration (e.g., glucose) by analog and/or digital means. In some cases, the signal processing module 148 can convert the current signal to a value representative of the analyte concentration by using a trans-impedance amplifier to convert the current to a voltage, an analog-to-digital converter (ADC) to convert this voltage to a numeric value, and further digital processing to generate an analyte concentration value.

### Fluid Reservoir

As further illustrated in FIG. 2A, fluid path within the pump base 140 conveys fluid from a fluid reservoir 144 through fluid path tube 145. In some embodiments, the fluid comprises a composition (e.g., insulin, insulin analog, etc.), a drug, or a medication. In some examples, the fluid can comprise glatiramer acetate, heparin, human menopausal gonadotropin, insulin, an insulin analog, a vitamin, a nutrient supplement, or any combination thereof. In some embodiments, the fluid can comprise at least one pharmaceutical acceptable excipient. In some cases, the at least one pharmaceutical acceptable excipient comprises phenol, cresol, a salts, a stabilizing agent, or any combination thereof.

In some embodiments, the fluid reservoir can hold about 2 mL to about 3 mL of fluid. In some embodiments, the fluid reservoir can hold about 2 mL to about 2.5 mL, about 2 mL to about 3 mL, or about 2.5 mL to about 3 mL of fluid. In some embodiments, the fluid reservoir can hold about 2 mL, about 2.5 mL, or about 3 mL of fluid. In some embodiments, the fluid reservoir can hold at least about 2 mL, or about 2.5 mL of fluid. In some embodiments, the fluid reservoir can hold at most about 2.5 mL, or about 3 mL of fluid. In some cases, the fluid reservoir can hold about 200 to about 300 units of insulin. In some embodiments, the fluid reservoir can hold about 200 units to about 250 units, about 200 units to about 300 units, or about 250 units to about 300 units of insulin. In some embodiments, the fluid reservoir can hold about 200 units, about 250 units, or about 300 unit of insulin. In some embodiments, the fluid reservoir can hold at least about 200 units, or about 250 units of insulin. In some embodiments, the fluid reservoir can hold at most about 250 units, or about 300 units of insulin. In some embodiments, the system comprises fluidic coupling of the cannula to a fluid reservoir to delivery fluid through a fluid path, as previously described. In some cases, the fluid path connector comprises a needle essentially parallel to the axis of the cylinder.

In some embodiments, the fluid path within the pump base 140 can convey fluid from the reservoir 144 through fluid path tube 145, driven by an electronic mechanism 142 (e.g., an electronic pump). In some embodiments, the electronic mechanism 142 comprises an electric motor-driven piston. In some cases, the electronic mechanism 142 comprises an internal power source. In some examples, the internal power source comprises a battery. In some cases, the electronic mechanism 142 comprises an external power source. The fluid path tube can further be 145 terminated at a septum 147 contained within a fluid path connector 143. The septum can be pierced by fluid path needle 114 on the sensing cannula assembly 100 during insertion completing the fluid path.

FIG. 2B, FIG. 2C, and FIG. 2D exemplary illustrate cross sections of the sensing cannula assembly attached to the pump following insertion, during which the assembly 100 can be advanced toward the pump base 140 in a direction parallel to the vertical axis of the assembly and the inserter needle 116. In some embodiments, the fluidic coupling in the system can be achieved by the fluid path needle 114 piercing the septum 147 during insertion, thereby forming a continuous fluid path from the pump 142 through fluid path 112. The fluid path can then enter the hollow core of sensing cannula 120 at the center of the sensing cannula assembly. Thus, in its final position, fluid can flow from the fluid reservoir 144, through fluid path tubing 145, into fluid path connector 143, through fluid path needle 114, and finally through fluid path tube 112 within the cannula. Once the pump is affixed to the skin and the assembly has been applied, fluid is then able to be delivered into the subcutaneous space of a subject.

Sensing Cannula

In some embodiments, the electrical coupling in the system comprises an electrode on the sensing cannula 120 operably connected to the signal processing electronics 148 through a plurality of conductors, as described herein. In some embodiments, the electrode on the sensing cannula 120 comprises an electrode for detecting an analyte concentration in the skin (e.g., subcutaneous space) of the subject. In some embodiments, the electrode is integrated into the sensing cannula 120. In some embodiments, the electrode is affixed to the sensing cannula 120 permanently or temporarily. In some embodiments, the electrode is affixed to the sensing cannula 120 by way of soldering, wire bonding, a conductive adhesive (e.g., silver epoxy), or mechanical pressure and potting compound. In some embodiments, the sensing cannula 120 comprises one, two, or three electrodes. In some embodiments, the sensing cannula 120 comprises at least one, two, or three electrodes. In some embodiments, the sensing cannula 120 comprises no more than one, two, or three electrodes.

In some embodiments, the electrode comprises a redox-catalytic layer. In some cases, the redox-catalytic layer comprises a redox mediator, an enzyme, or a combination thereof. In some instances, the redox mediator comprises a member of a redox mediator metal group. Examples of a redox mediator metal group may comprise metals in the platinum group. In some cases, the redox mediator metal comprises, by way of non-limiting examples, ruthenium, rhodium, palladium, osmium, iridium, and platinum. In some examples, the redox mediator metal group is covalently bound to a pyridine-based or an imidazole-based ligand. In some cases, the electrode is an amperometric glucose sensor. In some instances, the enzyme is an oxidoreductase. In some examples, the enzyme is glucose oxidase or glucose dehydrogenase. In such examples, the analyte is glucose.

The redox mediator and the enzyme in the redox-catalytic layer can allow electron transfer from an analyte in the subcutaneous space to the electrode. In some embodiments, the electron transfer is sufficient to cause a response to an applied bias potential of about −100 mV to about +250 mV relative to a reference electrode. In some cases, the response comprises a current. In some embodiments, the applied bias potential is about −100 mV to about −50 mV, about −100 mV to about 0 mV, about −100 mV to about +50 mV, about −100 mV to about +100 mV, about −100 mV to about +150 mV, about −100 mV to about +200 mV, about −100 mV to about +250 mV, about −50 mV to about 0 mV, about −50 mV to about +50 mV, about −50 mV to about +100 mV, about −50 mV to about +150 mV, about −50 mV to about +200 mV, about −50 mV to about +250 mV, about 0 mV to about +50 mV, about 0 mV to about +100 mV, about 0 mV to about +150 mV, about 0 mV to about +200 mV, about 0 mV to about +250 mV, about 50 mV to about +100 mV, about +50 mV to about +150 mV, about 50 mV to about +200 mV, about 50 mV to about +250 mV, about +100 mV to about +150 mV, about +100 mV to about +200 mV, about +100 mV to about +250 mV, about +150 mV to about +200 mV, about +150 mV to about +250 mV, or about +200 mV to about +250 mV relative to a reference electrode. In some embodiments, the applied bias potential is about −100 mV, about −50 mV, about 0 mV, about +50 mV, about +100 mV, about +150 mV, about +200 mV, or about +250 mV relative to a reference electrode. In some embodiments, the applied bias potential is at least about −100 mV, about −50 mV, about 0 mV, about +50 mV, about +100 mV, about +150 mV, or about +200 mV relative to a reference electrode. In some embodiments, the applied bias potential is at most about −50 mV, about 0 mV, about +50 mV, about +100 mV, about +150 mV, about +200 mV, or about +250 mV relative to a reference electrode.

In some embodiments, the electron transfer can be sufficient to cause a response to an applied bias potential of no more than about +250 mV relative to a reference electrode. In some cases, the response is a current. In some cases, the applied bias potential of no more than about +250 mV relative to the reference electrode allows the electrode layer to undergo substantially no electropolymerization of the excipient during continuous operation of the electrode. In such cases, the electrode can maintain a sensitivity to the analyte (e.g., glucose) in the presence of the composition (e.g., insulin or insulin analog).

In some embodiments, the sensing cannula 120 further comprises a conductor that is operably connected to the electrode described herein. In some embodiments, a signal from the electrode on the sensing cannula 120 is sent to the signal processing module 148 through a plurality of conductors, including at least one conductor on the sensing cannula 120. In some embodiments, the at least conductor is in physical contact with the electrode. In some embodiments, the at least one conductor is integrated into the sensing cannula 120. In some embodiments, the at least conductor is affixed to the sensing cannula 120 by way of soldering, wire bonding, conductive adhesive (e.g., silver epoxy), or mechanical pressure and potting compound. In some embodiments, the sensing cannula 120 comprises one, two, or three conductors. In some embodiments, the sensing cannula 120 comprises at least one, two, or three conductors. In some embodiments, the sensing cannula 120 comprises no more than one, two, or three conductors.

Referring to FIG. 2B, FIG. 2C, and FIG. 2D, in some embodiments, the electrical coupling in the system can be achieved by the one or more conductors 106 making contact with one or more electrical contacts 105 and 107 on the sensing cannula 120 within the assembly. Further, following insertion, the one or more conductors 106 can make contact with one or more conductors 132 retained within the ring 141 (shown in FIG. 2A). In some examples, the conductors 132 can be made of a compressible conductive rubber. In some cases, these conductors 132 can further be configured to be in electrical contact with the conductor 130. In some embodiments, the sensing cannula 120 comprises an electrode, as previously described herein. This can allow electrical currents flowing from an electrode on the sensing cannula 120 through the conductors on the sensing cannula 120 to enter conductor 106, which conveys the signals through conductors 132 and 130 and into the signal processing electronics 148 within the pump base 140.

In some embodiments, moisture can cause interference with currents developed by the sensor (e.g., electrode on the sensing cannula). In some embodiments, moisture can be prevented from entering the sensor contact area through the use of gasket 138. The gasket may be an O-ring composed of a compressible compound, such as, but not limited to, nitrile or silicone rubber. In some cases, this gasket 138 is compressed by the bottom surface of assembly 100. In some instances, the assembly 100 can be retained in the pump base 140 by a mechanism that may be a protrusion on the interior surface of retaining ring 141, by friction between the assembly 100 and the ring 141, or by a pressure sensitive adhesive.

Integrated Insertion Devices

In some embodiments, the device is configured to be driven into the skin using an insertion device (e.g., inserter), with the insertion device making temporary contact with the accessible surface of the body. In some embodiments, the insertion device provides for deployment of the sensing cannula into the skin of the subject. In some embodiments, the insertion device provides for deployment of the sensing cannula into the subcutaneous space of the subject. In some cases, the insertion device is permanently retained within a housing. In some instances, the entire insertion device is permanently retained within a housing. In alternative cases, the insertion device is not permanently retained within a housing.

In some embodiments, the insertion device has an insertion needle piercing a self-sealing inlet port, passing through a fluid delivery tube, and extending just beyond the distal end of the cannula, as illustrated in FIG. 2A-D. In some embodiments, the cannula has a fluid path formed by a permanently fixed needle that can be placed in the tissue (e.g., subcutaneous tissue), which remains in the tissue for the duration of use.

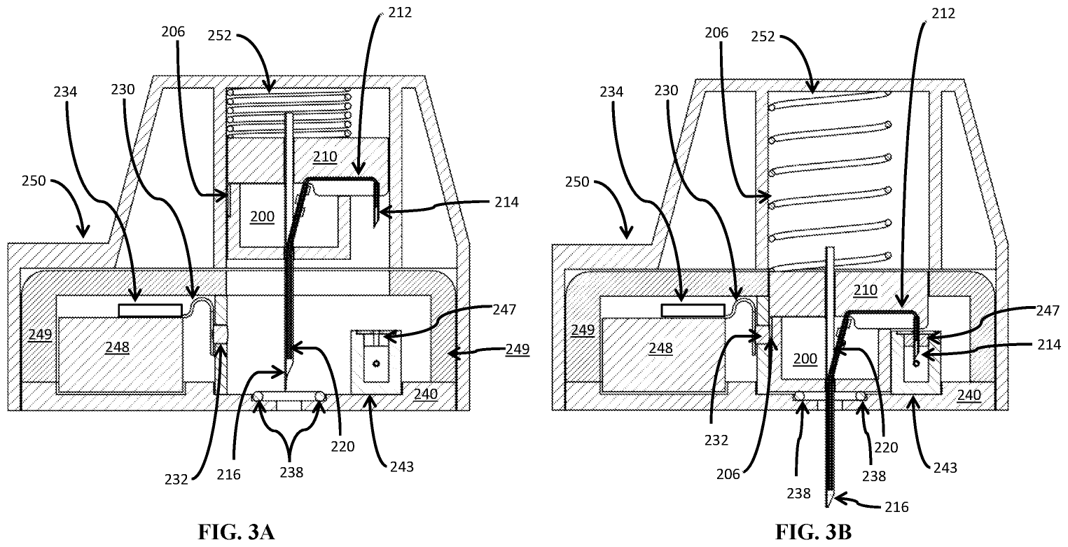

The system comprising an inserter is exemplary illustrated in FIG. 3A and FIG. 3B, which shows the interoperable sensing cannula assembly that is configured for attachment to a patch pump using a spring-loaded inserter 250. This spring-loaded inserter 250 can be powered by a spring 252, which is shown in its compressed state prior to deployment in FIG. 3A. The inserter 250 can retain the sensing cannula assembly 200 position prior to insertion. The inserter 250 can further be aligned to pump base 240 through the exterior vertical surfaces of the pump enclosure 249.

In some embodiments, the inserter can be released by pressing a button (not shown) that can withdraw a retaining feature from interfering with the outer wall of the sensing cannula assembly 200. With the pump base 240 adhered to the skin and the inserter held against the pump enclosure 249, the assembly 200 can then be accelerated quickly by the force of the spring 252. In some cases, a slight bump can be present on the side wall of assembly 200, which can slide past a raised feature in a retaining ring, allowing it to be held within the base 240. In such an embodiment, the sensing cannula 220 and the inserter needle 216 can be driven into the skin at a high speed.

The fluid path, as illustrated in FIG. 3B, can be completed upon a fluid path needle 214 piercing the septum 247. Further, the bottom of assembly 200 can be brought into contact with the pump base 240 and gasket 238, which can form a water-tight seal. Additionally, during insertion, the electrical circuit from sensing cannula 220 to signal processing electronics 248 is completed when a conductor 206 is brought into contact with the electrical contact 232. In some further embodiments, the inserter needle can then be removed, leaving the sensing cannula 220 placed in the subcutaneous tissue of a subject.

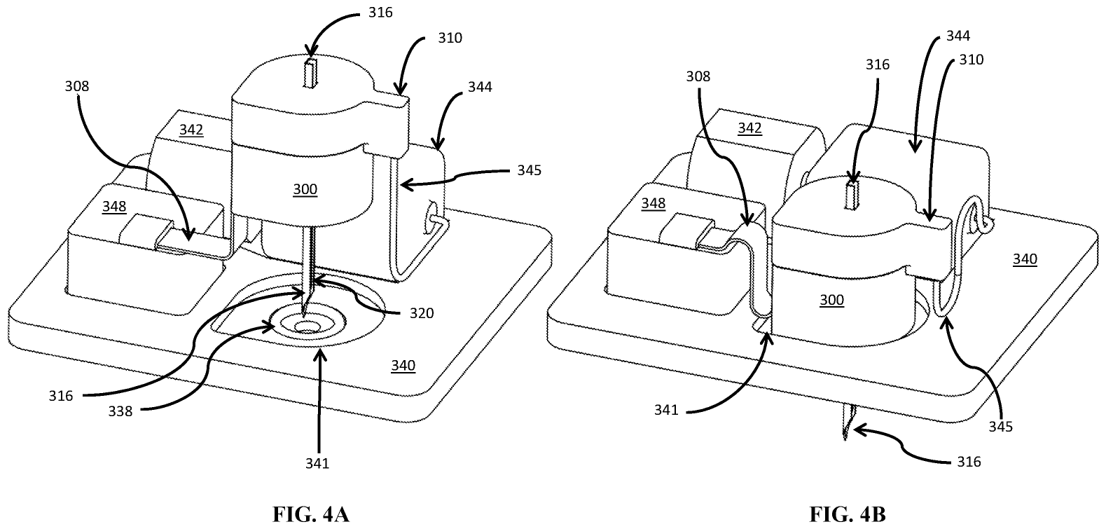

In an alternative embodiment, the electrical and fluid path contacts between the interoperable sensing cannula assembly and patch pump can be established during the assembly process, for example at a factory or facility during manufacturing. Such an embodiment comprising pre-established electrical and fluid path connections are exemplary illustrated in FIG. 4A and FIG. 4B. As shown, the sensing cannula assembly 300 comprising the sensing cannula 320 can be positioned within an inserter needle 316 and can be aligned to the pump base 340. In some embodiments, the system of FIG. 4A and FIG. 4B further comprises a pump enclosure, which is not illustrated herein for the sake of clarity.

In the present embodiment, the assembly can contain one or more electrical conductors 308 extending from the sensing cannula to the exterior surface of the housing and beyond, terminating at a socket 334 within the pump base 340. In some cases, these one or more electrical conductors 308 can have sufficient excess length (e.g., about 2 mm to about 15 mm), to allow for the movement of the assembly 300 during the insertion process. In some instances, the one or more electrical conductors 308 is about 2 mm to about 15 mm in length. In some instances, the one or more electrical conductors 308 about 2 mm to about 5 mm, about 2 mm to about 7 mm, about 2 mm to about 10 mm, about 2 mm to about 12 mm, about 2 mm to about 15 mm, about 5 mm to about 7 mm, about 5 mm to about 10 mm, about 5 mm to about 12 mm, about 5 mm to about 15 mm, about 7 mm to about 10 mm, about 7 mm to about 12 mm, about 7 mm to about 15 mm, about 10 mm to about 12 mm, about 10 mm to about 15 mm, or about 12 mm to about 15 mm in length. In some instances, the one or more electrical conductors 308 is about 2 mm, about 5 mm, about 7 mm, about mm, about 12 mm, or about 15 mm in length. In some instances, the one or more electrical conductors 308 is at least about 2 mm, about 5 mm, about 7 mm, about 10 mm, or about 12 mm in length. In some instances, the one or more electrical conductors 308 is at most about 5 mm, about 7 mm, about 10 mm, about 12 mm, or about 15 mm in length. These one or more electrical conductors 308 can be formed of conductive metals or alloys including, but not limited to, copper, brass, or steel. These one or more electrical conductors 308 can also be provided by a single or multi-conductor flexible electronic circuit, for example, a polyimide film supporting traces formed from patterned copper foil. The one or more conductors 308 can further enter the center of assembly 300 and terminate in the one or more contacts that are in electrical contact with the conductors on the cannula 320, as previously described.

Further, the length of fluid path tube 312 can be chosen to allow sufficient excess length (e.g., about 2 mm to about 15 mm), to allow for the displacement of the assembly 300 during insertion. In some cases, the fluid path tube 312 is about 2 mm to about 15 mm in length. In some cases, the fluid path tube 312 is about 2 mm to about 5 mm, about 2 mm to about 7 mm, about 2 mm to about 10 mm, about 2 mm to about 12 mm, about 2 mm to about 15 mm, about 5 mm to about 7 mm, about 5 mm to about 10 mm, about 5 mm to about 12 mm, about 5 mm to about 15 mm, about 7 mm to about 10 mm, about 7 mm to about 12 mm, about 7 mm to about 15 mm, about mm to about 12 mm, about 10 mm to about 15 mm, or about 12 mm to about 15 mm in length. In some cases, the fluid path tube 312 is about 2 mm, about 5 mm, about 7 mm, about 10 mm, about 12 mm, or about 15 mm in length. In some cases, the fluid path tube 312 is at least about 2 mm, about 5 mm, about 7 mm, about 10 mm, or about 12 mm in length. In some cases, the fluid path tube 312 is at most about 5 mm, about 7 mm, about 10 mm, about 12 mm, or about 15 mm in length. Within the fluid path arm 310, the fluid path tube 345 is extended into the sensing cannula to provide a continuous path from fluid reservoir 344. In some cases, this junction can be held in place by a friction fit or through the use of an adhesive. In some cases, the fluid path tube 345 can enter the sensing cannula 320 at the top or at the proximal end of the cannula. In some cases, the fluid path tube 345 can be continuous with the hollow core of the cannula to allow fluid to flow through the fluid path tube 345 into the cannula 320.

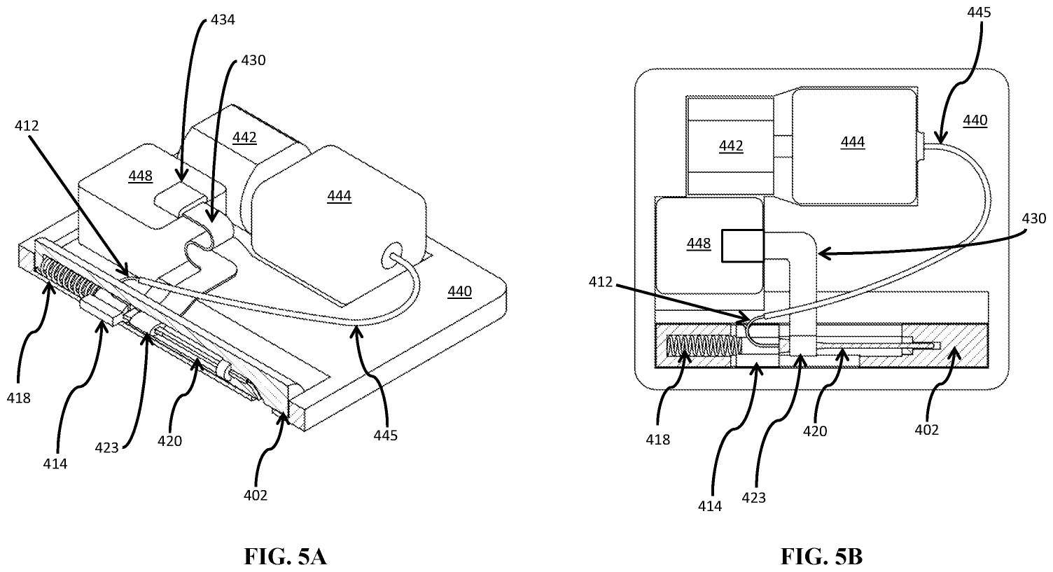

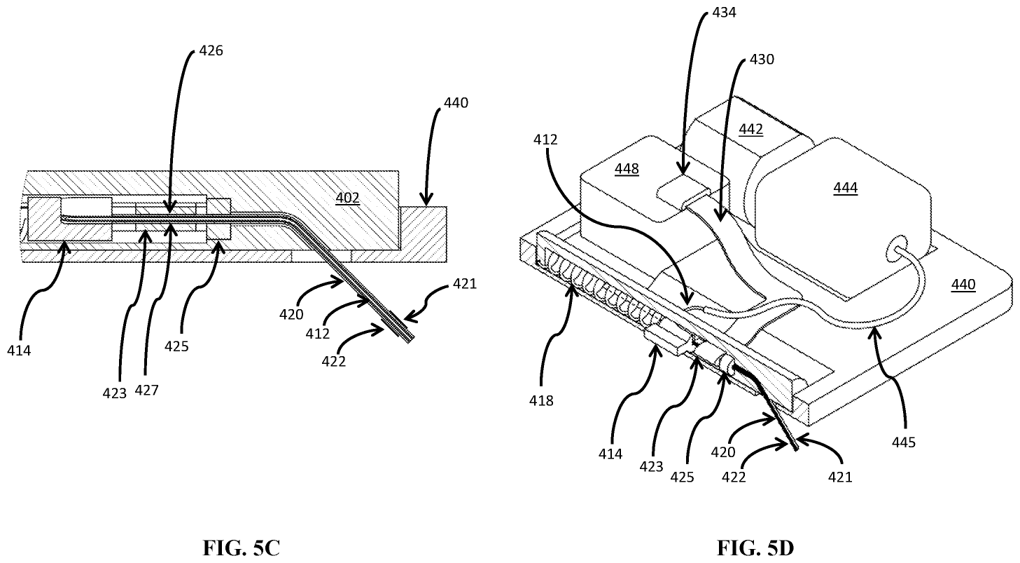

In a further alternative embodiment, the integrated sensing cannula assembly can have an integrated inserter mechanism fitted within the patch pump, which is exemplary illustrated in FIG. 1n some cases, the patch pump is a body-worn patch pump. FIG. 5A provides a perspective view of such system prior to insertion to the subcutaneous space, while FIG. 5B provides a top view of the same system. In some cases, the electrical path connections, the fluid path connections, or both, can be pre-established, for example, during manufacturing. In some instances, these pre-established connections can be made with sufficient excess length to permit the travel of the cannula during insertion without interrupting these connections.

In the embodiment of FIG. 5A and FIG. 5B, the sensing cannula assembly can be contained in an elongated structure such as housing 402, which can be placed into a base 440. In some cases, the housing 402 comprising the sensing cannula assembly can be placed into the base 440 during manufacturing. In some cases, prior to insertion, a conductor 430 can be in a folded configuration, providing electrical contact between the sensing cannula 420 and the signal processing electronics 448. This conductor 430 can be formed of conductive metals or alloys including, but not limited to copper, brass, or steel. This conductor 430 can further be provided by a single or multi-conductor flexible electronic circuit, for example, a polyimide film supporting traces formed from patterned copper foil. In some instances, the conductor 430 can be about 2 mm to about 15 mm in length. In some instances, the conductor 430 is about 2 mm to about 5 mm, about 2 mm to about 7 mm, about 2 mm to about 10 mm, about 2 mm to about 12 mm, about 2 mm to about 15 mm, about 5 mm to about 7 mm, about 5 mm to about 10 mm, about 5 mm to about 12 mm, about 5 mm to about 15 mm, about 7 mm to about 10 mm, about 7 mm to about 12 mm, about 7 mm to about 15 mm, about 10 mm to about 12 mm, about 10 mm to about 15 mm, or about 12 mm to about 15 mm in length. In some instances, the conductor 430 is about 2 mm, about 5 mm, about 7 mm, about 10 mm, about 12 mm, or about 15 mm in length. In some instances, the conductor 430 is at least about 2 mm, about 5 mm, about 7 mm, about 10 mm, or about 12 mm in length. In some instances, the conductor 430 is at most about 5 mm, about 7 mm, about 10 mm, about 12 mm, or about 15 mm in length. In some cases, the conductor 430 can be affixed to cannula 420 by means of a connector 423 that can hold the two components in contact. In some examples, the connector 423 can comprise an adhesive, a conductive adhesive, a plastic retaining clip, or any combination thereof.

Further, the fluid path can be formed by fluid path tubing 445 which can be connected to the sensing cannula tube 412. In some instances, this connection can comprise a slip fit of sensing cannula tube 412 into the pump fluid path, or a connector. In some examples, this connection may be strengthened with an adhesive. In some cases, the fluid path tube 445 is sufficiently long (e.g., about 5 mm to about 10 mm) and is sufficiently flexible to allow displacement of the connection to 412 by the distance required to deploy the sensor during insertion. In some cases, the fluid path tube 445 is about 2 mm to about 15 mm in length. In some cases, the fluid path tube 445 is about 2 mm to about 5 mm, about 2 mm to about 7 mm, about 2 mm to about 10 mm, about 2 mm to about 12 mm, about 2 mm to about 15 mm, about 5 mm to about 7 mm, about 5 mm to about 10 mm, about 5 mm to about 12 mm, about 5 mm to about 15 mm, about 7 mm to about 10 mm, about 7 mm to about 12 mm, about 7 mm to about 15 mm, about 10 mm to about 12 mm, about 10 mm to about 15 mm, or about 12 mm to about 15 mm in length. In some cases, the fluid path tube 445 is about 2 mm, about 5 mm, about 7 mm, about 10 mm, about 12 mm, or about 15 mm in length. In some cases, the fluid path tube 445 is at least about 2 mm, about 5 mm, about 7 mm, about 10 mm, or about 12 mm in length. In some cases, the fluid path tube 445 is at most about 5 mm, about 7 mm, about 10 mm, about 12 mm, or about 15 mm in length.

In some cases, the sensing cannula tube 412 can be retained within a sliding clip 414 that glides in a channel formed in the walls of the housing 402. In FIG. 5A and FIG. 5B, the spring 418 is shown in its compressed state, in which it applies force against sliding clip 414, which is held in place by interference from a catch (not shown). When the catch is retracted, the sliding clip 414 can be released and driven forward by the spring 418, driving sensing cannula 420 forward and into its final position (e.g., where the distal end is inserted into the subcutaneous tissue of a subject). In some instances, the sliding clip 414 can be released and can be driven forward rapidly by the spring 418. In some examples, the catch can be retracted by manual pressure on a hinged button, or by an electronic actuator, such as, but not limited to, a solenoid or thermal fuse material.

FIG. 5C and FIG. 5D exemplary illustrate a side section and a perspective view, respectively, of the present embodiment following insertion. As shown, the contents of the assembly can be advanced toward the edge of pump base 440 in a direction parallel to the horizontal axis of the assembly and the inserter needle (not shown). The sensing cannula fluid path 412 can then enter the hollow core of the cannula 420, which is held by the sliding clip 414. Once the cannula 420 is inserted, the conductor 430 can be in its fully extended state. In its extended state, the conductor 430 can make contact with electrical contacts 426 and 427 on sensing cannula 420 within the assembly. This can then allow electrical currents flowing from the sensor electrodes 421 and 422 through the conductors on the exterior surfaces of sensing cannula 420 to enter conductor 430, which conveys the signals to and from the signal processing electronics 448 within the pump base 440. In some cases, the electrodes 421 and 422 are integrated into the sensing cannula 420. In some cases, the electrode is affixed to the sensing cannula 420 permanently or temporarily. In some cases, the electrode is affixed to the sensing cannula 420 by way of soldering, wire bonding, or a conductive adhesive (e.g., silver epoxy). In some cases, the sensing cannula 420 comprises one, two, or three electrodes. In some cases, the sensing cannula 420 comprises at least one, two, or three electrodes. In some cases, the sensing cannula 420 comprises no more than one, two, or three electrodes. In some embodiments, the conductor on the exterior surface of the sensing cannula 420 is in physical contact with the one or more of the electrodes 421 and 422. In some embodiments, the conductor on the exterior surface of the sensing cannula 420 is integrated therein. In some embodiments, the conductor is affixed on the exterior surface of the sensing cannula 420 either permanently or temporarily. In some embodiments, the conductor on the exterior surface of the sensing cannula 420 is affixed to by way of soldering, wire bonding, or a conductive adhesive (e.g., silver epoxy). In some embodiments, the sensing cannula 420 comprises one, two, or three conductors. In some embodiments, the sensing cannula 420 comprises at least one, two, or three conductors. In some embodiments, the sensing cannula 420 comprises no more than one, two, or three conductors. Further, the conductor 430 can be held in electrical and mechanical contact with signal processing module 448 by means of a component such as a flex socket 434. In some cases, moisture can be prevented from entering the sensor contact area through the use of gasket 425.

In some cases, the sensing cannula tube 412 can be retracted following insertion, with the sensing cannula 420 held in place by a connector 423, while the tube 412 is withdrawn by the sliding action of the sliding clip 414. In some instances, this can be driven by the release of a second spring (not shown). In some examples, this movement can be triggered by the movement of the sliding clip 414 to depress an actuator as it is driven by the first spring 418.

Methods of Making the Interoperable Sensing Cannula

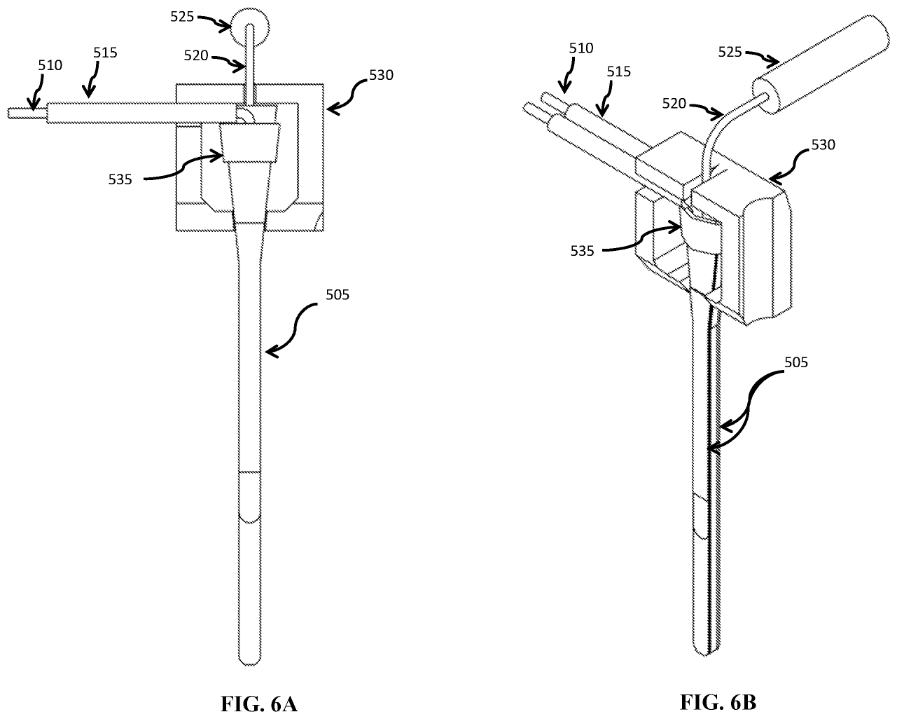

In some embodiments, the sensing cannula as described herein can be fabricated by laminating layers of a material around a mandrel (e.g., solid, or hollow tubing). An exemplary sensing cannula that can be fabricated via this method is illustrated in FIG. 6A and FIG. 6B. In some embodiments, the sensing cannula comprises one or more metal electrodes 505 forming planar surfaces on opposite sides of the sensing cannula.

In some embodiments, the fluid path 520 can be formed by removing the solid mandrel. In some alternative embodiments, the fluid path 520 can be formed by leaving the hollow tubing inside of the lamination. In some cases, the hollow tubing can be partially retracted from the lamination, thereby exposing some of the interior of the cannula to act as a portion of the fluid path 520. In some embodiments, the fluid path 520 comprises a fluid path tubing 525 on the exterior. In some cases, the fluid path tubing comprises a flexible polymer. In some examples, the flexible polymer comprises a thermoplastic. In some examples, the flexible polymer comprises PTFE, FEP, or any combination thereof. In some examples, a flexible polymer, such as PTFE, FEP, or a combination thereof, can line the inside of a tubing composed of another thermoplastic polymer. In some embodiments, the fluid path 520 and the fluid path tubing 525 can be further extended and connected to a fluid reservoir in order to deliver a fluid through the sensing cannula, as described herein.

In some embodiments, the interoperable assembly is made by attaching conductors to the electrode surfaces. In some cases, the conductors can comprise one or more of a solid or stranded wire, flex circuit material, ribbon cable, or can be extensions of the electrode layer beyond the core of the cannula. In some examples, the conductors comprise one or more wires 510. In some embodiments, the attachment of the conductors can be made by soldering, conductive adhesive, wire bonding, or mechanical pressure with the aid of potting compound. In some cases, the one or more wires 510 comprises insulation 515 on their exterior. In some cases, the one or more wires 510 extends to an electrical contact. In some examples, the one or more wires 510 is in electrical contact with a conductor coupled to a signal processing module, as described herein. In some examples, the electrical contact is on the housing of the interoperable sensing cannula assembly, as described herein.

In some embodiments, the portion of the sensing cannula where the electrical and fluid connection is made can be held in place by a ring member 535. In some cases, the ring member 535 comprise a non-conductive material. In some cases, the ring member 535 comprises a rigid or heat-shrink tubing. In some instances, the ring member 535 comprises a non-conductive polymer. In some examples, the non-conductive polymer is polyolefin. In some embodiments, the portion of the sensing cannula comprising the electronic circuit and fluid connections are further potted inside of a plastic shell 530 which constrains the geometry of the potted volume. In some embodiments, the portion of the sensing cannula comprising the electronic circuit and fluid connections does not comprise a plastic shell 530. In some cases, potting the electronic circuit and fluid connections helps to maintain the integrity of the electrical connection, the fluid connection, or a combination thereof.

Methods of Using the Interoperable Sensing Cannula

In some embodiments, the interoperable sensing cannula assembly, as described herein, can be inserted into the subcutaneous space of a subject to treat a disease or disorder, as illustrated, for example, in embodiments of FIG. 3A-B, FIG. 4A-B, and FIG. 5A-D. The interoperable sensing cannula assembly can be used for sensing an analyte concentration and delivering a composition to a subcutaneous space in a subject to treat a disease or disorder by providing a sensing cannula, inserting the sensing cannula into the subcutaneous space, monitoring a behavior of the analyte concentration in the subcutaneous space, and delivering a sufficient amount of the composition through the sensing cannula into the subcutaneous space in response to the behavior of the analyte concentration. In some embodiments, the sensing cannula comprises an electrode for detecting the analyte concentration in the subcutaneous space of the subject. In some examples, the sensing cannula comprises a plurality of electrodes (e.g., two electrodes). In some cases, the electrode is on the external surface of the sensing cannula. In some cases, detecting the analyte concentration comprises measuring an applied bias potential of the electrode relative to a reference electrode, as described herein. In some cases, the electrode comprises a redox-catalytic layer comprising a redox mediator and an enzyme, as described herein. In some examples, the disease or disorder comprises insulin resistance. In some examples, the disease or disorder comprises a Type 1 diabetes mellitus. In some examples, the disease or disorder comprises a Type 2 diabetes mellitus.

In some embodiments, the assembly can be used for in vivo monitoring of analyte concentrations including, but not limited to, pH, oxygen, lactate, glucose, or insulin concentration. In some embodiments, analyte concentration monitoring can have applications in various situations including, but not limited to, treatment of multiple sclerosis, fertility treatments, diabetes, nutritional supplementation, and automated drug dosing. In some embodiments, the assembly can be used to deliver a composition, drug, or medication including but not limited to glatiramer acetate, heparin, human menopausal gonadotropin, insulin, an insulin analog, a vitamin, a nutrient supplement, or any combination thereof. In some cases, the composition comprises fast-acting insulin, intermediate-acting insulin, long-acting insulin, or any combination thereof. In some embodiments, the composition further comprises at least one pharmaceutical acceptable excipient. In some cases, the at least one pharmaceutical acceptable excipient comprises phenol, cresol, a salts, a stabilizing agent, or any combination thereof.

In some cases, the sensing cannula can monitor a behavior of the analyte concentration in the subcutaneous space. In some cases, a sufficient amount of the composition can be delivered through the sensing cannula into the subcutaneous space in response to the behavior of the analyte concentration. In some instances, the behavior of the analyte concentration comprises an analyte concentration reaching a threshold. In some examples, the analyte concentration comprises glucose concentration (e.g., in the subcutaneous space of a subject). In some examples, the electrode comprises an amperometric glucose sensor. In some examples, the threshold of the glucose concentration comprises about 40 mg/dL to about 400 mg/dL. In some examples, the glucose concentration comprises about 40 mg/dL to about 100 mg/dL, about 40 mg/dL to about 125 mg/dL, about 40 mg/dL to about 150 mg/dL, about 40 mg/dL to about 175 mg/dL, about 40 mg/dL to about 200 mg/dL, about 40 mg/dL to about 250 mg/dL, about 40 mg/dL to about 300 mg/dL, about 40 mg/dL to about 350 mg/dL, about 40 mg/dL to about 400 mg/dL, about 100 mg/dL to about 125 mg/dL, about 100 mg/dL to about 150 mg/dL, about 100 mg/dL to about 175 mg/dL, about 100 mg/dL to about 200 mg/dL, about 100 mg/dL to about 250 mg/dL, about 100 mg/dL to about 300 mg/dL, about 100 mg/dL to about 350 mg/dL, about 100 mg/dL to about 400 mg/dL, about 125 mg/dL to about 150 mg/dL, about 125 mg/dL to about 175 mg/dL, about 125 mg/dL to about 200 mg/dL, about 125 mg/dL to about 250 mg/dL, about 125 mg/dL to about 300 mg/dL, about 125 mg/dL to about 350 mg/dL, about 125 mg/dL to about 400 mg/dL, about 150 mg/dL to about 175 mg/dL, about 150 mg/dL to about 200 mg/dL, about 150 mg/dL to about 250 mg/dL, about 150 mg/dL to about 300 mg/dL, about 150 mg/dL to about 350 mg/dL, about 150 mg/dL to about 400 mg/dL, about 175 mg/dL to about 200 mg/dL, about 175 mg/dL to about 250 mg/dL, about 175 mg/dL to about 300 mg/dL, about 175 mg/dL to about 350 mg/dL, about 175 mg/dL to about 400 mg/dL, about 200 mg/dL to about 250 mg/dL, about 200 mg/dL to about 300 mg/dL, about 200 mg/dL to about 350 mg/dL, about 200 mg/dL to about 400 mg/dL, about 250 mg/dL to about 300 mg/dL, about 250 mg/dL to about 350 mg/dL, about 250 mg/dL to about 400 mg/dL, about 300 mg/dL to about 350 mg/dL, about 300 mg/dL to about 400 mg/dL, or about 350 mg/dL to about 400 mg/dL. In some examples, the glucose concentration comprises about 40 mg/dL, about 100 mg/dL, about 125 mg/dL, about 150 mg/dL, about 175 mg/dL, about 200 mg/dL, about 250 mg/dL, about 300 mg/dL, about 350 mg/dL, or about 400 mg/dL. In some examples, the glucose concentration comprises at least about 40 mg/dL, about 100 mg/dL, about 125 mg/dL, about 150 mg/dL, about 175 mg/dL, about 200 mg/dL, about 250 mg/dL, about 300 mg/dL, or about 350 mg/dL. In some examples, the glucose concentration comprises at most about 100 mg/dL, about 125 mg/dL, about 150 mg/dL, about 175 mg/dL, about 200 mg/dL, about 250 mg/dL, about 300 mg/dL, about 350 mg/dL, or about 400 mg/dL. In some examples, the behavior of the analyte concentration comprises a duration that the analyte concentration remains at a threshold, such as those described herein.

In some further instances, the behavior of the analyte concentration comprises a derivative of the analyte concentration. In some examples, the behavior of the analyte concentration comprises a first order derivative of the analyte concentration (e.g., rate of change of the analyte concentration, for example, in mg·dL⁻¹·min⁻¹). In some examples, the rate of change in the analyte concentration comprises an increase in the analyte concentration. In some examples, the rate of change in the analyte concentration comprises a decrease in the analyte concentration. In some examples, the rate f change in the analyte concentration can comprise about 1 mg·dL⁻¹·min⁻¹ to about 10 mg·dL⁻¹·min⁻¹. In some examples, the rate of change in the analyte concentration can comprise about 1 mg·dL⁻¹·min⁻¹ to about 2 mg·dL⁻¹·min⁻¹, about 1 mg·dL⁻¹·min⁻¹ to about 3 mg·dL⁻¹·min⁻¹, about 1 mg·dL⁻¹·min⁻¹ to about 4 mg·dL⁻¹·min⁻¹, about 1 mg·dL⁻¹·min⁻¹ to about 5 mg·dL⁻¹·min⁻¹, about 1 mg·dL⁻¹·min⁻¹ to about 6 mg·dL⁻¹·min⁻¹, about 1 mg·dL⁻¹·min⁻¹ to about 7 mg·dL⁻¹·min⁻¹, about 1 mg·dL⁻¹·min⁻¹ to about 8 mg·dL⁻¹·min⁻¹, about 1 mg/dL/min to about 9 mg·dL⁻¹·min⁻¹, about 1 mg·dL⁻¹·min⁻¹ to about 10 mg·dL⁻¹·min⁻¹, about 2 mg·dL⁻¹·min⁻¹ to about 3 mg·dL⁻¹·min⁻¹, about 2 mg·dL⁻¹·min⁻¹ to about 4 mg·dL⁻¹·min⁻¹, about 2 mg·dL⁻¹·min⁻¹ to about 5 mg·dL⁻¹·min⁻¹, about 2 mg·dL⁻¹·min⁻¹ to about 6 mg·dL⁻¹·min⁻¹, about 2 mg·dL⁻¹·min⁻¹ to about 7 mg·dL⁻¹·min⁻¹, about 2 mg·dL⁻¹·min⁻¹ to about 8 mg·dL⁻¹·min⁻¹, about 2 mg·dL⁻¹·min⁻¹ to about 9 mg·dL⁻¹·min⁻¹, about 2 mg·dL⁻¹·min⁻¹ to about 10 mg·dL⁻¹·min⁻¹, about 3 mg·dL⁻¹·min⁻¹ to about 4 mg·dL⁻¹·min⁻¹, about 3 mg·dL⁻¹·min⁻¹ to about 5 mg·dL⁻¹·min⁻¹, about 3 mg·dL⁻¹·min⁻¹ to about 6 mg·dL⁻¹·min⁻¹, about 3 mg·dL⁻¹·min⁻¹ to about 7 mg·dL⁻¹·min⁻¹, about 3 mg·dL⁻¹·min⁻¹ to about 8 mg·dL⁻¹·min⁻¹, about 3 mg·dL⁻¹·min⁻¹ to about 9 mg·dL⁻¹·min⁻¹, about 3 mg·dL⁻¹·min⁻¹ to about 10 mg·dL⁻¹·min⁻¹, about 4 mg·dL⁻¹·min⁻¹ to about 5 mg·dL⁻¹·min⁻¹, about 4 mg·dL⁻¹·min⁻¹ to about 6 mg·dL⁻¹·min⁻¹, about 4 mg·dL⁻¹·min⁻¹ to about 7 mg·dL⁻¹·min⁻¹, about 4 mg·dL⁻¹·min⁻¹ to about 8 mg·dL⁻¹·min⁻¹, about 4 mg·dL⁻¹·min⁻¹ to about 9 mg·dL⁻¹·min⁻¹, about 4 mg·dL⁻¹·min⁻¹ to about 10 mg·dL⁻¹·min⁻¹, about 5 mg·dL⁻¹·min⁻¹ to about 6 mg·dL⁻¹·min⁻¹, about 5 mg·dL⁻¹·min⁻¹ to about 7 mg·dL⁻¹·min⁻¹, about 5 mg·dL⁻¹·min⁻¹ to about 8 mg·dL⁻¹·min⁻¹, about 5 mg·dL⁻¹·min⁻¹ to about 9 mg·dL⁻¹·min⁻¹, about 5 mg·dL⁻¹·min⁻¹ to about 10 mg·dL⁻¹·min⁻¹, about 6 mg·dL⁻¹·min⁻¹ to about 7 mg·dL⁻¹·min⁻¹, about 6 mg·dL⁻¹·min⁻¹ to about 8 mg·dL⁻¹·min⁻¹, about 6 mg·dL⁻¹·min⁻¹ to about 9 mg·dL⁻¹·min⁻¹, about 6 mg·dL⁻¹·min⁻¹ to about 10 mg·dL⁻¹·min⁻¹, about 7 mg·dL⁻¹·min⁻¹ to about 8 mg·dL⁻¹·min⁻¹, about 7 mg·dL⁻¹·min⁻¹ to about 9 mg·dL⁻¹·min⁻¹, about 7 mg·dL⁻¹·min⁻¹ to about 10 mg·dL⁻¹·min⁻¹, about 8 mg·dL⁻¹·min⁻¹ to about 9 mg·dL⁻¹·min⁻¹, about 8 mg·dL⁻¹·min⁻¹ to about 10 mg·dL⁻¹·min⁻¹, or about 9 mg·dL⁻¹·min⁻¹ to about 10 mg·dL⁻¹·min⁻¹. In some examples, the rate of change in the analyte concentration can comprise about 1 mg·dL⁻¹·min⁻¹, about 2 mg·dL⁻¹·min⁻¹, about 3 mg·dL⁻¹·min⁻¹, about 4 mg·dL⁻¹·min⁻¹, about 5 mg·dL⁻¹·min⁻¹, about 6 mg·dL⁻¹·min⁻¹, about 7 mg·dL⁻¹·min⁻¹, about 8 mg·dL⁻¹·min⁻¹, about 9 mg·dL⁻¹·min⁻¹, or about 10 mg·dL⁻¹·min⁻¹. In some examples, the rate of change in the analyte concentration can comprise at least about 1 mg·dL⁻¹·min⁻¹, about 2 mg·dL⁻¹·min⁻¹, about 3 mg·dL⁻¹·min⁻¹, about 4 mg·dL⁻¹·min⁻¹, about 5 mg·dL⁻¹·min⁻¹, about 6 mg·dL⁻¹·min⁻¹, about 7 mg·dL⁻¹·min⁻¹, about 8 mg·dL⁻¹·min⁻¹, or about 9 mg·dL⁻¹·min⁻¹. In some examples, the rate of change in the analyte concentration can comprise at most about 2 mg·dL⁻¹·min⁻¹, about 3 mg·dL⁻¹·min⁻¹, about 4 mg·dL⁻¹·min⁻¹, about 5 mg·dL⁻¹·min⁻¹, about 6 mg·dL⁻¹·min⁻¹, about 7 mg·dL⁻¹·min⁻¹, about 8 mg·dL⁻¹·min⁻¹, about 9 mg·dL⁻¹·min⁻¹, or about 10 mg·dL⁻¹·min⁻¹. In some further instances, the behavior of the analyte concentration comprises a second order, third order, or fourth order derivative of the analyte concentration (e.g., mg·dL⁻¹·min⁻², mg·dL⁻¹·min⁻³, mg·dL⁻¹·min⁻⁴). In some further instances, the behavior of the analyte concentration comprises a duration that the analyte concentration changes at a given rate or a derivative thereof, such as those described herein.

Once the base and the assembly is affixed to the skin, the sensing cannula can be inserted into the subcutaneous space of a subject via an inserter, as previously described herein. In some embodiments, when inserted into the subcutaneous space of a subject, the sensing cannula projects outward from the skin contact surface at an angle between about 40 degrees to about 60 degrees. In some embodiments, the cannula projects outward from the skin contact surface at an angle between about 40 degrees to about 50 degrees, about 40 degrees to about 60 degrees, or about 50 degrees to about 60 degrees. In some embodiments, the cannula projects outward from the skin contact surface at an angle between about 40 degrees, about 50 degrees, or about 60 degrees. In some embodiments, the cannula projects outward from the skin contact surface at an angle between at least about 40 degrees, or about 50 degrees. In some embodiments, the cannula projects outward from the skin contact surface at an angle between at most about 50 degrees, or about 60 degrees. In some embodiments, the cannula projects outward from the skin contact surface essentially perpendicularly. In some embodiments, the cannula projects outward from the skin contact surface at an angle of about 90 degrees.

Once inserted on the subcutaneous space, fluid can be delivered to the subcutaneous space from a fluid reservoir, through a fluid path tube, and further flow through the fluid path tube within the cannula, as previously described herein. In some embodiments, fluid can be delivered to a subject for about 3 days to about 10 days. In some embodiments, fluid can be delivered to a subject for about 3 days to about 5 days, about 3 days to about 7 days, about 3 days to about 10 days, about 5 days to about 7 days, about 5 days to about 10 days, or about 7 days to about 10 days. In some embodiments, fluid can be delivered to a subject for about 3 days, about 5 days, about 7 days, or about 10 days. In some embodiments, fluid can be delivered to a subject for at least about 3 days, about 5 days, or about 7 days. In some embodiments, fluid can be delivered to a subject for at most about 5 days, about 7 days, or about 10 days. For example, if the system comprises a glucose sensor and a fluid reservoir comprising insulin or an insulin analog, the system of the present disclosure can allow the subject to avoid the pain and inconvenience of mealtime needle sticks to measure blood glucose during that timeframe.

### EXAMPLES

The following illustrative examples are representative of embodiments of the systems and methods described herein and are not meant to be limiting in any way.

### Example 1—Interoperable Sensing Cannula Assembly with Inserter

The system comprising an interoperable sensing cannula and an insertion device is illustrated in FIG. 3A and FIG. 3B. The insertion device is a spring-loaded inserter 250 comprising a spring 252, which is aligned to a pump base 240 through the exterior vertical surfaces of the pump enclosure 249. The system further comprises an insertion needle 216 for piercing the skin of a subject, as well as a sensing cannula 220 for sensing an analyte concentration in the skin of the subject and delivering a fluid thereto.

The system shown in FIG. 3A is placed on the skin of a subject. The inserter 250 is released by pressing a button that withdraws a retaining feature from interfering with the outer wall of the sensing cannula assembly 200. With the pump base 240 adhered to the skin and the inserter 250 held against the pump enclosure 249, the assembly 200 is then accelerated quickly by the force of the spring 252. A slight bump present on the side wall of assembly 200 slides past a raised feature in a retaining ring, allowing it to be held within the base 240.

The fluid path, as illustrated in FIG. 3B, is completed when the fluid path needle 214 pierces the septum 247. Further, the bottom of assembly 200 is brought into contact with the pump base 240 and gasket 238, which forms a water-tight seal. Additionally, the electrical circuit from sensing cannula 220 to signal processing electronics 248 is completed as the conductor 206 is brought into contact with the electrical contact 232. The inserter needle 216 is then removed, leaving the sensing cannula 220 placed in the subcutaneous tissue of a subject.

### Example 2—Pre-Connected Interoperable Sensing Cannula Assembly

The electrical circuit and the fluid path of the system described in Example 1 is pre-connected during manufacturing of the device, as shown in FIG. 4A and FIG. 4B.

The assembly 300 contains one or more electrical conductors 308 extending from the sensing cannula 320 to the exterior surface of the housing and beyond, and terminating at a socket 334 within the pump base 340. The one or more electrical conductors 308 have sufficient excess length, between about 5 mm to about 10 mm, to allow for the movement of the assembly 300 during the insertion process. The one or more electrical conductors 308 are formed of conductive metals or alloys. The one or more conductors 308 further enters the center of assembly 300 and terminates in one or more contacts that are in electrical contact with the conductors on cannula 320.

The length of fluid path tube 312 is about 5 mm to about 10 mm to have sufficient length for the displacement of the assembly 300 during insertion. Within the fluid path arm 310, fluid path tube 345 is extended through the sensing cannula 320 to provide a continuous path from fluid reservoir 344. This junction is held in place by a friction fit or through the use of an adhesive. The fluid path tube 345 enters the sensing cannula 320 at the top or at the proximal end of the cannula 320, and is in continuous flow with the hollow core of the cannula to allow fluid to flow from the fluid path tube 345 into the cannula 320 once the device is deployed on the skin surface of a subject, as previously described in Example 1.

### Example 3—Pre-Connected Interoperable Sensing Cannula Assembly with Integrated Inserter

The inserter device is integrated into the assembly, in addition to providing pre-connected electronic circuit and fluid connections, as described in Example 2. As described in Example 2, sufficient excess length is provided in both the electronic circuit and fluid connections to permit the travel of the cannula during insertion without interrupting these connections.

As shown in FIG. 5A and FIG. 5B, the sensing cannula assembly is contained in an elongated housing 402, which is placed into base 440 during manufacturing. Further, prior to insertion, the conductor 430 is in a folded configuration, providing electrical contact from the sensing cannula 420 into the signal processing electronics 448. The conductor 430 is affixed to cannula 420 by a connector 423 that holds the two components in contact. Further, the fluid path tubing 445 is connected to the sensing cannula tube 412 via a slip fit of the tube 412 into the fluid path tubing 445. This connection can further comprise a connector or an adhesive to further strengthen the connection.

The sensing cannula tube 412 is retained within a sliding clip 414 that glides in a channel formed in the walls of housing 402. The spring 418 is in its compressed state prior to deployment, and the sliding clip 414 is held in place by interference from a catch. Upon deployment, the spring 418 applies force against the sliding clip 414 and the catch is retracted, so that the sliding clip 414 is released and driven forward by the spring 418. This mechanism drives the sensing cannula 420 forward, so that the distal end is inserted into the subcutaneous tissue of a subject.

Once deployed, as shown in FIG. 5C and FIG. 5D, the contents of the assembly are advanced toward the edge of pump base 440 in a direction parallel to the horizontal axis of the assembly and an inserter needle. The sensing cannula fluid path 412 enters the hollow core of the cannula 420 held by the sliding clip 414. The conductor 430 is in its fully extended state, and is in contact with electrical contacts 426 and 427 on sensing cannula 420 within the assembly. This allows electrical currents flowing from the sensor electrodes 421 and 422 through the conductors on the exterior surfaces of sensing cannula 420 to enter conductor 430, which conveys the signals to and from the signal processing electronics 448 within the pump base. The conductor 430 is held in electrical and mechanical contact with signal processing module 448 by means of a flex socket 434. Further, moisture is prevented from entering the sensor contact area through the gasket 425.

The sensing cannula tube 412 is then retracted following insertion. This is done by holding the sensing cannula 420 in place by connector 423, while the tube 412 is withdrawn by the sliding action of sliding clip 414.

### Example 4—Interoperable Sensing Cannula Assembly for Glucose Sensing

The devices of Example 1, Example 2, or Example 3 are configured to detect glucose concentration in the subcutaneous space of a subject with an insulin resistance disorder (e.g., Type 1 diabetes mellitus, Type 2 diabetes mellitus, etc.). The electrode on the sensing cannula is an amperometric glucose sensor with a redox mediator and an enzyme. The redox mediator is osmium or ruthenium that is covalently bound to a pyridine-based or an imidazole-based ligand. The enzyme is a glucose oxidoreductase (e.g., glucose oxidase or glucose dehydrogenase). The fluid reservoir comprises an insulin or insulin analog composition.

The device described in Example 1, Example 2, or Example 3 are placed on the skin of a subject and the sensing cannula is inserted into the subcutaneous space via an inserter that drives the inserter needle to pierce the skin of the subject. Once the sensing cannula is inserted, the inserter needle is removed, leaving only the sensing cannula within the subcutaneous tissue.

The redox mediator and the enzyme of the electrode allows electron transfer from glucose in the subcutaneous space to the electrode. As the electrons flow from the glucose to the electrode, a current is generated that is proportional to the glucose concentration in the subcutaneous tissue. This current is received by the signal processing module, which sends power to an electronic mechanism (e.g., an electric motor-driven piston) that drives fluid flow from the fluid reservoir. The fluid from the fluid reservoir flows through the fluid path tube and the sensing cannula, and into the subcutaneous tissue, thereby delivering the insulin composition to the subject.

If the electron transfer is sufficient to cause a response, in the form of a current, to an applied bias potential of no more than about +250 mV relative to the reference electrode, the electrode layer undergoes substantially no electropolymerization and maintains a sensitivity to glucose in the presence of the composition comprising insulin or an insulin analog.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1.-93. (canceled)

94. A device for sensing an analyte concentration and delivering a composition into a subcutaneous space, comprising:
a. a sensing cannula comprising an electrode for detecting the analyte concentration in the subcutaneous space, wherein the electrode comprises a redox-catalytic layer;
b. a signal processing module operably coupled to the sensing cannula through a plurality of conductors; and
c. a fluid reservoir configured to deliver the composition through the sensing cannula into the subcutaneous space upon receiving a signal from the signal processing module.

95. The device of claim 94, further comprising a housing having a top surface, a base, and one or more vertical walls.

96. The device of claim 95, wherein the housing further comprises one or more electrical contacts on the one or more vertical walls.

97. The device of claim 95, wherein one or more of the plurality of conductors is contained in the housing.

98. The device of claim 95, wherein one or more of the plurality of conductors is disposed on an external surface of the one or more vertical walls of the housing.

99. The device of claim 94, wherein the sensing cannula further comprises one or more electrical contacts.

100. The device of claim 99, wherein the one or more of the plurality of conductors is in contact with the one or more electrical contacts on the sensing cannula.

101. The device of claim 99, wherein one or more of the plurality of conductors is disposed on an exterior surface of the sensing cannula.

102. The device of claim 94, wherein one or more of the plurality of conductors is operably coupled to the electrode.

103. The device of claim 94, wherein one or more of the plurality of conductors is held in contact with the sensing cannula through a connector.

104. The device of claim 94, further comprising a fluid path connecting the fluid reservoir and the sensing cannula.

105. The device of claim 94, wherein the redox-catalytic layer comprises a redox mediator and an enzyme.

106. The device of claim 105, wherein the redox mediator comprises a metal in a platinum group that is covalently bound to a pyridine-based or imidazole-based ligand.

107. The device of claim 105, wherein the enzyme comprises glucose oxidase or glucose dehydrogenase.

108. The device of claim 105, wherein the redox mediator and the enzyme allow electron transfer from an analyte in the subcutaneous space to the electrode.

109. The device of claim 108, wherein the electron transfer is sufficient to cause a response to an applied bias potential to the electrode of no more than +250 millivolts (mV) relative to a reference electrode.

110. The device of claim 109, wherein the response comprises a current.

111. The device of claim 109, wherein the applied bias potential to the electrode of no more than +250 mV relative to the reference electrode allows the electrode layer to undergo substantially no electropolymerization of an excipient of the composition during continuous operation of the electrode, thereby maintaining a sensitivity of the electrode to analyte in the presence of the composition.

112. The device of claim 94, wherein the electrode comprises an amperometric glucose sensor.

113. The device of claim 94, wherein the analyte comprises glucose.

114. The device of claim 94, wherein the composition comprises insulin or an insulin analog.

115. The device of claim 94, wherein the composition further comprises a pharmaceutical acceptable excipient.

116. The device of claim 115, wherein the pharmaceutical acceptable excipient comprises phenol, cresol, a salt, a stabilizing agent, or any combination thereof.

117. The device of claim 94, wherein the plurality of conductors comprises a conductive metal or alloy.

118. The device of claim 94, wherein the plurality of conductors comprises a compressible conductive rubber.

119. The device of claim 94, wherein the plurality of conductors comprises a flexible electronic circuit.

120. The device of claim 94, further comprising a spring configured to drive the sensing cannula into the subcutaneous space.

121. A device for sensing analyte concentration and delivering a composition into a subcutaneous space, the device comprising:
a. a sensing cannula comprising an electrode for detecting an analyte concentration in the subcutaneous space;
b. a signal processing module operably coupled to the sensing cannula through a plurality of conductors; and
c. an electronic pump configured to depress a syringe fluidically connected to a fluid reservoir upon receiving a signal from the signal processing module, wherein depressing the syringe delivers the composition through the sensing cannula into the subcutaneous space.

122. A method for sensing an analyte concentration and delivering a composition into a subcutaneous space, comprising:
a. providing a sensing cannula, wherein the sensing cannula comprises an electrode for detecting the analyte concentration in the subcutaneous space, wherein the electrode comprises a redox-catalytic layer;
b. inserting the sensing cannula into the subcutaneous space;
c. monitoring, by the sensing cannula, the analyte concentration in the subcutaneous space; and
d. delivering a sufficient amount of the composition through the sensing cannula into the subcutaneous space based at least in part on the monitoring of the analyte concentration.
